(19) United States
(12) Patent Application Publication
(10) Pub. No.: US 2005/0034224 A1
(43) Pub. Date: Feb. 17, 2005

(54) Swimming mask

(76) Inventor: Chih-Cheng Shiue, Rancho Santa Fe, CA (US)

Correspondence Address: BRUCE H. TROXELL, SUITE 1404, 5205 LEESBURG PIKE, FALLS CHURCH, VA, 22041, US

(21) Appl. No.: 10/681,240

(22) Filed: Oct. 9, 2003

(30) Foreign Application Priority Data
Aug. 14, 2003 (TW) 92214738

(51) Int. Cl. A61F 9/02

(52) U.S. Cl. 2/428

(57) ABSTRACT

The present invention is relating to the improvement of swimming mask, especially to a swimming mask having two bands in both sides of mask body formed integrally. Furthermore, the band adjustment is in the rear portion of head. By the way, the structure of swimming mask can be simplified and the facial pressure can be more even.

### FIELD OF THE INVENTION

The present invention relates to the improvement of the swimming mask, especially to the body of swimming mask provided for activity of swimming, diving, and scuba diving. By the present invention the mask body of the diving mask is integrally formed with the strings or bands that act as the mask strap to secure the mask on the user's face.

### BACKGROUND OF THE INVENTION

Usually, conventional facial masks for swimming can be divided to two categories, namely framed structure type and frameless structure type. Said framed structure type mask, the mask frame is made by stiff or rigid material and the lens are mounted into the mask body and mask frame. Furthermore, the buckles with bands are connected with both sides of mask frame. Other than framed structure type mask, the frameless structure type mask, the mask body is combined directly with the lens and the buckles with bands are connected on the both sides of mask body.

The above-mentioned two basic types of mask body are made of soft or resilient material, such as silicon rubber. The main purpose of using soft material is to cover the face tightly to prevent the water flow into the space between thereof.

However, due to the material of frame being stiff and solid, when the band is tightly pulled, the pressure through mask body to the face is uneven. As to the frameless mask, the pressure through the mask body to the face is easily influenced by the adjustment of buckles on both sides and thus subject to also being uneven.

Therefore, the above-mentioned two basic structures have the common disadvantage, that is, the structure and the design of the entire mask are restricted by the buckle as well as relating components and it could be further improved.

Currently in the market, there is a kind of mask which provides the adjustment of the band on the back of head portion. The mask is attached to two bands by buckles on both sides of mask frame or mask body. Although it is somewhat reduces the complication on both sides of the mask body and mask frame, we believe it could be further improved.

### SUMMARY OF THE INVENTION

The present invention is providing a mask having two bands, one on each sides of mask body. Said bands are formed integrally with mask body.

The above-mentioned structure allows the both sides of mask body to have a simplified shape and structure.

The ends of above-mentioned two bands can be connected or join together.

The ends of above-mentioned two bands can be positioned in the adjustment device in the back portion of head and further to provide the fasten function of the mask.

### BRIEF DESCRIPTION OF THE DRAWINGS

FIG. 1 is the perspective view of the embodiment of the present invention.

FIG. 2 is the top view of the embodiment of the present invention.

FIG. 3 is the right side view of the embodiment of the present invention.

### DETAILED DESCRIPTION OF PREFERRED EMBODIMENTS

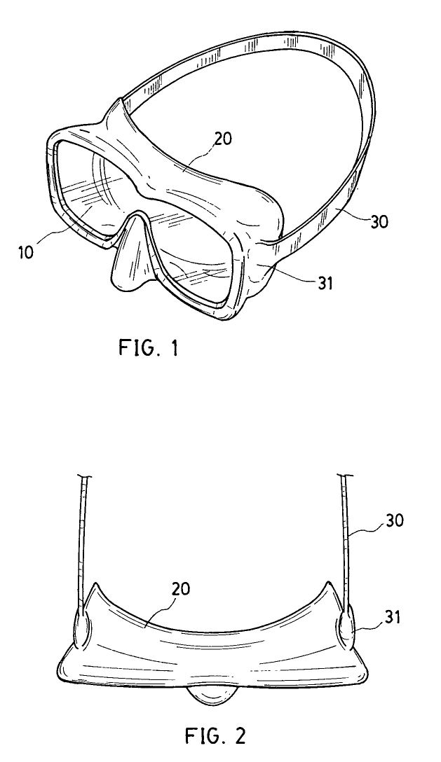

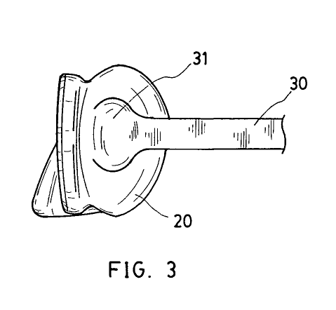

Refer to FIG. 1, 2 & 3, they show the embodiment of the frameless mask of the present invention. It includes a lens 10 and a mask body 20. Both sides of mask body 20 provide the band 30.

Said lens 10 is not limited to single lens but also can be two lens as well as multi-lens.

Said mask body 20 also can be designed as having frame and not limited to the frameless structure.

Said bands 30 are not limited to the type showing on the drawing, the end of bands 30 may provide the device can be adjusted and the device is not limited to a specified type.

Mask body 20 and bands 30 can be formed by mold injection integrally to obtain the better connection between thereof.

The area of the connection portion 31 of mask body 20 and bands 30 can be enlarged to share the pulling tension of bands and make the pressure to the face more gentle.

In conclusion, through mold injection integrally, the tension of the mask of the present invention can be predetermined and allow the designer having the freedom of design for entire mask.

1) The improvement of swimming mask, characterized that the resilient bands are formed integrally to both sides of mask body, thereby simplifying the structure between mask body and bands.

2) The improvement of swimming mask as claimed in claim 1, the joint portion of between mask body and bands are enlarged.

3) The improvement of swimming mask as claimed in claim 1, the ends of said bands can be connected integrally.

4) The improvement of swimming mask as claimed in claim 1, the ends of said bands can be forked into two string.
